(19) United States
(12) Patent Application Publication
(10) Pub. No.: US 2023/0041725 A1
(43) Pub. Date: Feb. 9, 2023

(54) DETERMINATION DEVICE

(71) Applicant: NEC Solution Innovators, Ltd., Koto-ku, Tokyo (JP)

(72) Inventor: Kazuhide UMEDA, Tokyo (JP)

(73) Assignee: NEC Solution Innovators, Ltd., Koto-ku, Tokyo (JP)

(21) Appl. No.: 17/791,645

(22) PCT Filed: Dec. 28, 2020
(86) PCT No.: PCT/JP2020/049165

§ 371 Date: Jul. 8, 2022

(30) Foreign Application Priority Data
Jan. 30, 2020 (JP) 2020-013982

(51) Int. Cl. G06V 40/40; G06V 40/18; G06V 10/75; G06V 10/26; G06V 10/60; G06V 10/145

(52) U.S. Cl. CPC G06V 40/40; G06V 10/26; G06V 10/60; G06V 10/75; G06V 10/145; G06V 40/18

(57) ABSTRACT

A determination device includes an acquisition unit that acquires image data with a flash and an image data without a flash, the image data with a flash being captured in a state of using a flash, the image data without a flash being image data of an imaging object that is the same as the imaging object shown in the image data with a flash and being captured in a state of not using a flash, and a determination unit that determines whether or not the imaging object is a living body by comparing an eye area of the imaging object included in the image data with a flash with an eye area of the imaging object included in the image data without a flash.

### TECHNICAL FIELD

The present invention relates to a determination device, a determination method, and a storage medium.

### BACKGROUND ART

Authentication technologies such as face authentication and iris authentication have been known. In the authentication such as face authentication and iris authentication, it is known that there are problems of impersonation and erroneous authentication using image data.

As a technology of suppressing erroneous authentication in the case of using image data, Patent Literature 1 has been known for example. Patent Literature 1 describes a method including a step of acquiring an image captured by irradiating a subject with light coaxially with the optical axis of a camera, and a second step of determining whether or not the eye captured in the image is an eye of a living body on the basis of a luminance value in the pupil area of the eye. According to Patent Literature 1, a first image captured by irradiating a subject with light coaxially with the optical axis of the camera, and a second image captured by irradiating the subject with light with an optical axis that is different from the optical axis of the camera, are acquired. Then, it is determined whether or not the eye in the first image and the second image is an eye of a living body on the basis of the luminance value in the pupil area of the eye, by using the fact that retinal reflex is caused in the eye of a living body.

Patent Literature 1: JP 2006-326326 A

### SUMMARY

In the art described in Patent Literature 1, when capturing an object only once, there is a possibility of erroneously authenticate an image in which light has been reflected on an eye. Further, in the art of Patent Literature 1, in the case of capturing an object twice, it is necessary to capture the object by irradiating the object with light coaxially with the optical axis of the camera and also capture the object by irradiating the object with light with an optical axis that is different from the optical axis of the camera. Therefore, a plurality of lighting devices are required. This causes a problem that it is difficult to suppress erroneous authentication in the case of using photographs, with a simple configuration.

In view of the above, an object of the present invention is to provide a determination device, a determination method, and a storage medium capable of solving a problem that it is difficult to suppress erroneous authentication in the case of using photographs or the like with a simple configuration.

In order to achieve such an object, a determination device according to one aspect of the present invention is configured to include
an acquisition unit that acquires image data with a flash and image data without a flash, the image data with a flash being captured in a state of using a flash, the image data without a flash being image data of an imaging object that is same as an imaging object shown in the image data with a flash and being captured in a state of not using a flash; and
a determination unit that determines whether or not the imaging object is a living body by comparing an eye area of the imaging object included in the image data with a flash with an eye area of the imaging object included in the image data without a flash.

Further, a determination method according to another aspect of the present invention is configured to include, by an information processing device,
acquiring image data with a flash and image data without a flash, the image data with a flash being captured in a state of using a flash, the image data without a flash being image data of an imaging object that is same as an imaging object shown in the image data with a flash and being captured in a state of not using a flash; and
determining whether or not the imaging object is a living body by comparing an eye area of the imaging object included in the acquired image data with a flash with an eye area of the imaging object included in the acquired image data without a flash.

Further, a storage medium according to another aspect of the present invention is a computer-readable storage medium storing thereon a program for causing an information processing device to realize processing to:

acquire image data with a flash and image data without a flash, the image data with a flash being captured in a state of using a flash, the image data without a flash being image data of an imaging object that is same as an imaging object shown in the image data with a flash and being captured in a state of not using a flash; and
determine whether or not the imaging object is a living body by comparing an eye area of the imaging object included in the acquired image data with a flash with an eye area of the imaging object included in the acquired image data without a flash.

With the configurations described above, the present invention is able to provide a determination device, a determination method, and a storage medium that enable suppression of erroneous authentication in the case of using photographs or the like, with a simple configuration.

### BRIEF DESCRIPTION OF DRAWINGS

FIG. 1 is a block diagram illustrating an exemplary configuration of an information processing device according to a first exemplary embodiment of the present invention.

FIG. 2 illustrates an exemplary arrangement of an imaging unit and a screen display unit.

FIG. 3 illustrates an example of an eye part in image data with a flash.

FIG. 4 illustrates an example of an eye part in image data without a flash.

FIG. 5 illustrates examples of image data with a flash and image data without a flash in the cases of an eye of a living body and a photograph.

FIG. 6 is a flowchart illustrating an exemplary operation of the information processing device according to the first exemplary embodiment of the present invention.

FIG. 7 is a block diagram illustrating an exemplary configuration of an information processing device according to a second exemplary embodiment of the present invention.

FIG. 8 illustrates an example of a flash pattern.

FIG. 9 illustrates an example of a flash pattern in image data.

FIG. 10 is an illustration for explaining exemplary processing of an extraction unit and a comparison and determination unit.

FIG. 11 is a flowchart illustrating an exemplary operation of the information processing device according to the second exemplary embodiment of the present invention.

FIG. 12 illustrates another example of a flash pattern.

FIG. 13 illustrates an exemplary hardware configuration of a determination device according to a third exemplary embodiment of the present invention.

FIG. 14 is a block diagram illustrating an exemplary configuration of a determination device.

FIG. 15 is a block diagram illustrating another exemplary configuration of a determination device.

### EXEMPLARY EMBODIMENTS

### First Exemplary Embodiment

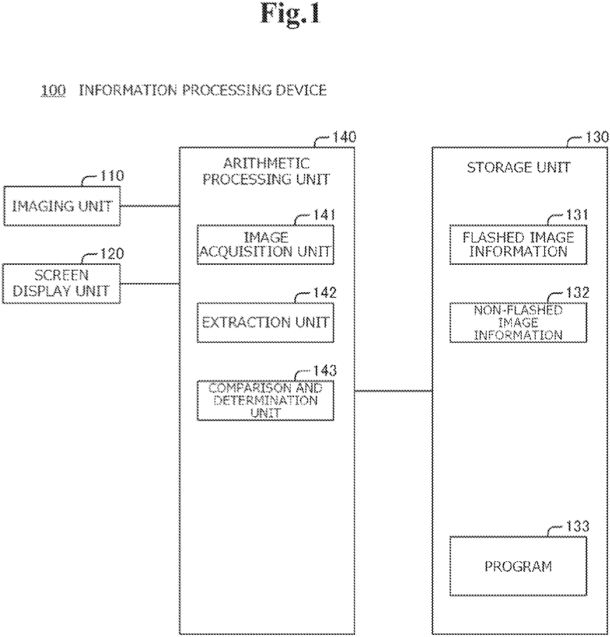

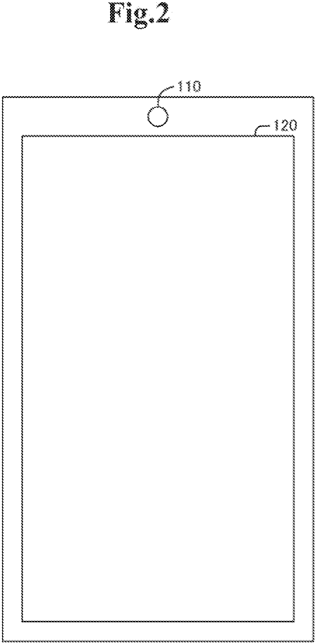

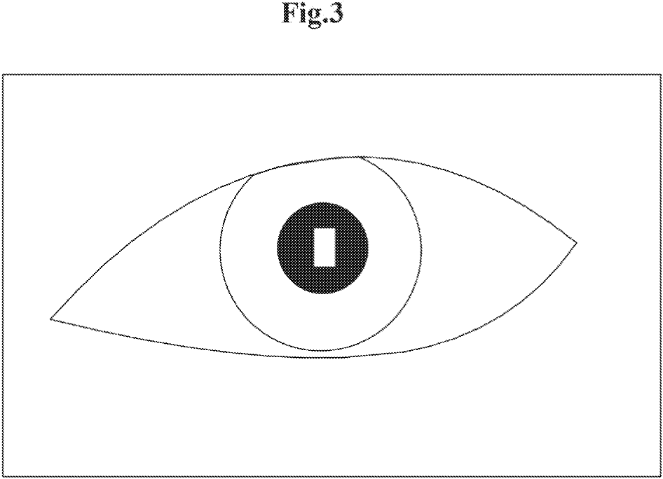

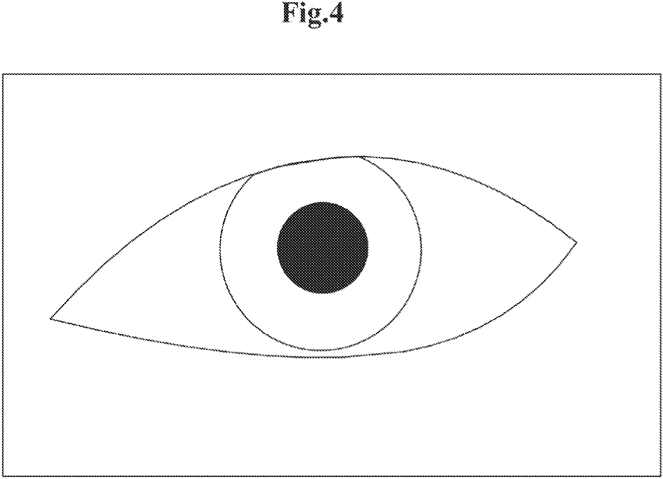

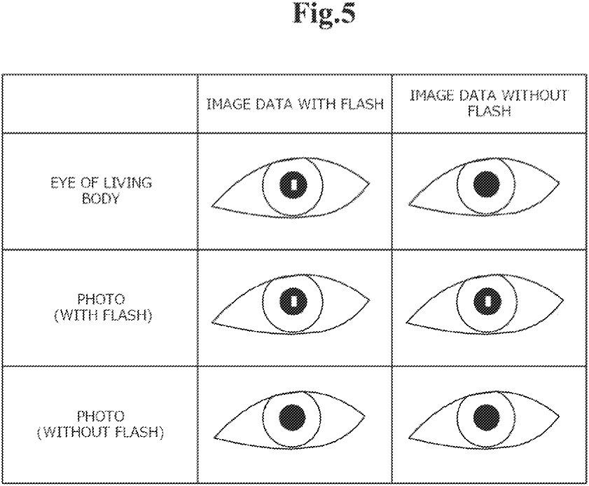

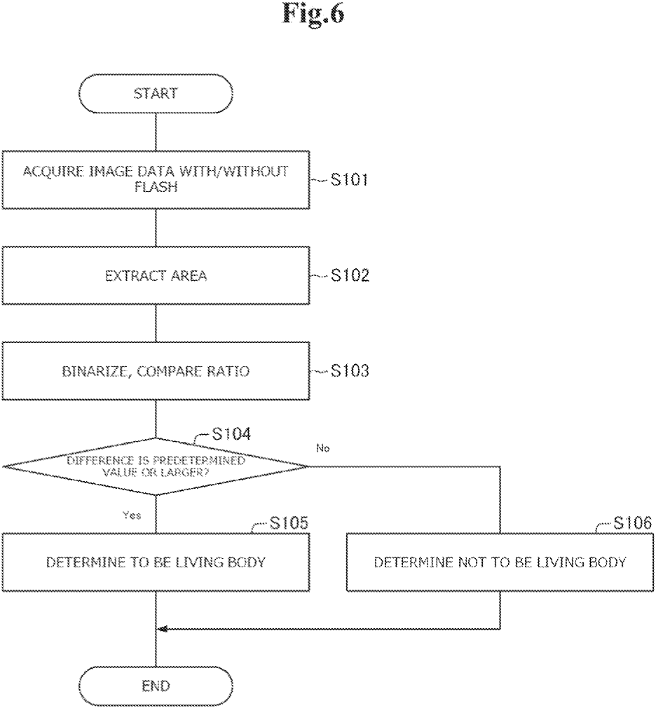

A first exemplary embodiment of the present invention will be described with reference to FIGS. 1 to 6. FIG. 1 is a block diagram illustrating an exemplary configuration of an information processing device 100. FIG. 2 illustrates an exemplary arrangement of an imaging unit 110 and a screen display unit 120. FIG. 3 illustrates an example of an eye part in image data with a flash. FIG. 4 illustrates an example of an eye part in image data without a flash. FIG. 5 illustrates examples of image data with a flash and image data without a flash in the cases of an eye of a living body and a photograph. FIG. 6 is a flowchart illustrating an exemplary operation of the information processing device 100.

The first exemplary embodiment of the present invention describes the information processing device 100 (determination device) that determines whether an imaging object is a living body or one other than a living body such as a photograph, on the basis of image data in which a face of a person is captured. As described below, the information processing device 100 acquires, when acquiring image data, image data with a flash that is captured using a flash and image data without a flash that is captured not using a flash. Then, the information processing device 100 compares the acquired image data with a flash and image data without a flash to thereby determine whether or not the imaging object is a living body.

Note that image data such as image data with a flash and image data without a flash can be used for authentication such as face authentication and iris authentication, for example. Image data such as image data with a flash and image data without a flash may be used for a purpose other than authentication.

The information processing device 100 is a device that acquires image data. The information processing device 100 of the present embodiment is a mobile terminal such as a smartphone. The information processing device 100 may be a device other than a mobile terminal illustrated as an example.

FIG. 1 illustrates an exemplary configuration of the information processing device 100. Referring to FIG. 1, the information processing device 100 includes an imaging unit 110, a screen display unit 120, a storage unit 130, and an arithmetic processing unit 140, as constituent elements.

The imaging unit 110 acquires image data according to an instruction from the image acquisition unit 141. When capturing image data, the imaging unit 110 can use a flash function using the screen display unit 120 described below, a clock function of the information processing device 100, or the like. For example, the imaging unit 110 may be a camera previously installed in the information processing device 100.

As described below, the imaging unit 110 can capture image data in a state of using a flash and capture image data in a state of not using a flash continuously, according to an instruction of the image acquisition unit 141. That is, the imaging unit 110 can capture image data with a flash and image data without a flash continuously at predetermined intervals.

The screen display unit 120 is a display device that displays an image according to an instruction from the arithmetic processing unit 140. The screen display unit 120 may be a touch panel that detects an operation of an operator performed on the screen display unit 120 and outputs it to the arithmetic processing unit 140.

The screen display unit 120 can function as a flash by increasing the luminance value of an image to be displayed on the screen display unit 120, when the imaging unit 110 captures image data. For example, the screen display unit 120 can function as a flash by, for example, displaying a white image in which the luminance value is set to be higher than a predetermined value (for example, to be a highest value that can be set), according to an instruction by the image acquisition unit 141.

FIG. 2 illustrates an exemplary arrangement of the imaging unit 110 and the screen display unit 120. For example, as illustrated in FIG. 2, the imaging unit 110 and the screen display unit 120 are arranged on the same plane of the information processing device 100. For example, in the case of FIG. 2, the imaging unit 110 is disposed above the screen display unit 120 in a rectangular shape. Note that the imaging unit 110 and the screen display unit 120 may be in a position relationship other than that illustrated in FIG. 2, as long as they are in a position relationship in which a flash is reflected on the eye of the imaging object when image data is captured with a flash.

The storage unit 130 is a storage device such as a hard disk or a memory. The storage unit 130 stores therein processing information and a program 133 required for various types of processing executed in the arithmetic processing unit 140. The program 133 is read and executed by the arithmetic processing unit 140 to thereby implement various processing units. The program 133 is read in advance from an external device or a storage medium via the data input/output function of the information processing device 100, and is stored in the storage unit 130. Main information to be stored in the storage unit 130 includes, for example, flashed image information 131 and non-flashed image information 132.

The flashed image information 131 includes image data with a flash that is image data captured using a flash by the imaging unit 110. In other words, the flashed image information 131 includes image data with a flash captured by the imaging unit 110 in a state where the luminance value of an image shown on the screen display unit 120 is increased. For example, in the flashed image information 131, image data with a flash and information indicating the time that the image data with a flash was captured by the imaging unit 110 are associated with each other.

In the position relation between the imaging unit 110 and the screen display unit 120 as illustrated in FIG. 2 for example, when a living body is captured with a flash, the flash is reflected on the eye part in the image data with a flash as illustrated in FIG. 3. For example, when the screen display unit 120 is in a rectangular shape, as illustrated in FIG. 3, a flash in a rectangular shape is reflected on the captured image data (image data with a flash).

The non-flashed image information 132 includes image data without a flash that is image data captured not using a flash by the imaging unit 110. For example, in the non-flashed image information 132, image data without a flash and information indicating the time that the image data without a flash was captured by the imaging unit 110 are associated with each other.

When a living body is captured without a flash, no flash is reflected on the eye part as illustrated in FIG. 4, which is different from the case of using a flash described with reference to FIG. 3.

The arithmetic processing unit 140 includes a microprocessor such as an MPU and the peripheral circuits thereof, and is configured to read and execute the program 133 from the storage unit 130 to allow the hardware and the program 133 to cooperate with each other to thereby implement the various processing units. Main processing units implemented by the arithmetic processing unit 140 include, for example, an image acquisition unit 141, an extraction unit 142, and a comparison and determination unit 143.

The image acquisition unit 141 instructs the imaging unit 110 to capture image data, and acquires image data from the imaging unit 110. The image acquisition unit 141 also controls a flash using the screen display unit 120.

For example, the image acquisition unit 141 instructs the imaging unit 110 to continuously capture two types of image data, that is, image data with a flash and image data without a flash. The image acquisition unit 141 acquires image data with a flash and also acquires image data without a flash from the imaging unit 110. Then, the image acquisition unit 141 stores the acquired image data with a flash in the storage unit 130, in association with the time that the image data with a flash was captured by the imaging unit 110, as the flashed image information 131. The image acquisition unit 141 also stores the acquired image data without a flash in the storage unit 130, in association with the time that the image data without a flash was captured by the imaging unit 110, as the non-flashed image information 131.

For example, the image acquisition unit 141 acquires an image data pair consisting of image data with a flash and image data without a flash, as described above.

The extraction unit 142 extracts at least a pupil area of an eye part from image data such as an image data with a flash and an image data without a flash. For example, the extraction unit 142 extracts an area other than the white of an eye, such as an iris area or a pupil area, from image data such as an image data with a flash or an image data without a flash.

For example, when extracting the predetermined area, the extraction unit 142 can use a processing result of face authentication processing. According to the face authentication processing, the center point of a pupil can be taken for example. Therefore, the extraction unit 142 can be configured to extract, for example, a circular area having a predetermined size from the center point of the pupil specified by face authentication processing to thereby extract the area. The extraction unit 142 may be configured to extract a predetermined area on the basis of color information such as RGB, for example.

The comparison and determination unit 143 compares predetermined areas extracted from image data with a flash and image data without a flash, that are an image data pair, respectively by the extraction unit 142. For example, the comparison and determination unit 143 performs binarization processing of black and white on the respective areas extracted by the extraction unit 142. Then, on the basis of a result of comparing a result of binarizing the image data with a flash and a result of binarizing the image data without a flash, the comparison and determination unit 143 determines whether or not the imaging object of the image data with a flash and the image data without a flash, that are an image data pair, is a living body.

For example, the comparison and determination unit 143 calculates the ratio of black and while in the area extracted from the image data with a flash and a ratio of black and white in the area extracted from the image data without a flash. For example, the comparison and determination unit 143 calculates at least one of the ratio of white in each area and the ratio of black in each area. Then, on the basis of a result of comparing the calculated ratios, the comparison and determination unit 143 determines whether or not the capturing object is a living body. Specifically, for example, when the difference between the ratio of black and white in the area extracted from image data with a flash and the ratio of black and white in the area extracted from image data without a flash is equal to or larger than a predetermined comparative threshold, the comparison and determination unit 143 determines that the captured object is a living body. On the contrary, when the difference between the ratio of black and white in the area extracted from image data with a flash and the ratio of black and white in the area extracted from image data without a flash is smaller than a predetermined comparative threshold, the comparison and determination unit 143 determines that the captured object is not a living body.

In the case where an eye of a living body is captured, as illustrated in FIG. 5, a flash is reflected on image data with a flash but a flash is not reflected on image data without a flash. Therefore, between the image data with a flash and the image data without a flash, the ratio of black and white when the area is binarized varies. On the other hand, in the case of a photograph having been captured with a flash, a flash is reflected in both the image data with a flash and the image data without a flash. Further, in the case of a photograph having been captured without a flash, a flash is not reflected in both the image data with a flash and the image data without a flash. That is, in the case of an imaging object other than a living body such as using a photograph, no difference is caused between image data with a flash and image data without a flash. With the relationship as described above, when the comparison and determination unit 143 performs comparison between image data with a flash and image data without a flash as described above, it is possible to easily determine whether or not the imaging object is a living body.

Note that in the present embodiment, specific values of a threshold for binarizing areas and a comparative threshold are not limited specifically. Any values may be set to a threshold for binarization and a comparative threshold. Further, each of a threshold for binarization and a comparative threshold may be a preset fixed value or a value that varies flexibly. For example, a comparative threshold may be determined according the size of the screen display unit 120 and the magnitude of a flash.

The exemplary configuration of the information processing device 100 is as described above. Next, an exemplary operation of the information processing device 100 will be described with reference to FIG. 6.

Referring to FIG. 6, the image acquisition unit 141 of the information processing device 100 acquires a pair of image data with a flash and image data without a flash captured continuously (step S101).

The extraction unit 142 extracts at least a pupil area of an eye part from image data such as an image data with a flash or an image data without a flash (step S102). For example, the extraction unit 142 extracts an area other than the white of an eye, such as an iris area or a pupil area, from image data such as an image data with a flash or an image data without a flash. When extracting the predetermined area, the extraction unit 142 may use a processing result of face authentication processing.

The comparison and determination unit 143 compares the areas extracted, by the extraction unit 142, from image data with a flash and image data without a flash that are an image data pair. For example, the comparison and determination unit 143 performs binarization processing of black and white on the respective areas extracted by the extraction unit 142. Then, the comparison and determination unit143 compares a result of binarizing the image data with a flash and a result of binarizing the image data without a flash (step S103).

Specifically, for example, when the difference between the ratio of black and white in the area extracted from the image data with a flash and the ratio of black and white in the area extracted from the image data without a flash is equal to or larger than a predetermined comparative threshold (step S104, Yes), the comparison and determination unit 143 determines that the imaging object is a living body (step S105). On the other hand, when the difference between the ratio of black and white in the area extracted from the image data with a flash and the ratio of black and white in the area extracted from the image data without a flash is smaller than a predetermined comparative threshold (step S104, No), the comparison and determination unit 143 determines that the imaging object is not a living body (step S106).

The exemplary operation of the information processing device 100 is as described above.

As described above, the information processing device 100 includes the comparison and determination unit 143. Further, the information processing device 100 is configured to acquire image data with a flash and image data without a flash. With this configuration, the comparison and determination unit 143 can determine whether or not the imaging object is a living body on the basis of a comparison result between image data with a flash and image data without a flash. Thereby, it is possible to easily determine that the object is not a living body when image data such as a photograph is imaged and to suppress erroneous authentication, with a simple configuration.

The present embodiment has been described the case where the information processing device 100 includes the imaging unit 110. However, the information processing device 100 may be configured to acquire image data from an external camera or the like having a flash function. That is, the information processing device 100 may not have a function as the imaging unit 110. Further, the present embodiment has described the case where the screen display unit 120 is used as a flash. However, a function as a flash may be implemented by that other than the screen display unit 120.

Further, the present embodiment has described the case where one information processing device has the respective functions as illustrated in FIG. 1. However, in the present invention, a plurality of information processing devices may have the respective functions illustrated in FIG. 1 distributively. For example, when the information processing device 100 is a mobile terminal such as a smartphone, the information processing device 100 may be configured to transmit image data with a flash and image data without a flash, captured by the information processing device 100, to an external information processing device such as an external determination device. In the case of such a configuration, the external information processing device may have functions as the extraction unit 142 and the comparison and determination unit 143, for example.

Note that the present invention can be used for a case other than the case of using a mobile terminal such as a smartphone, like performing entrance/exit management and open/close management of a gate and a delivery locker by using face authentication, iris authentication, and the like. The present invention may be used for determining whether or not an imaging object is a living body in any scene other than that illustrated above (for example, other than the authentication purpose).

### Second Exemplary Embodiment

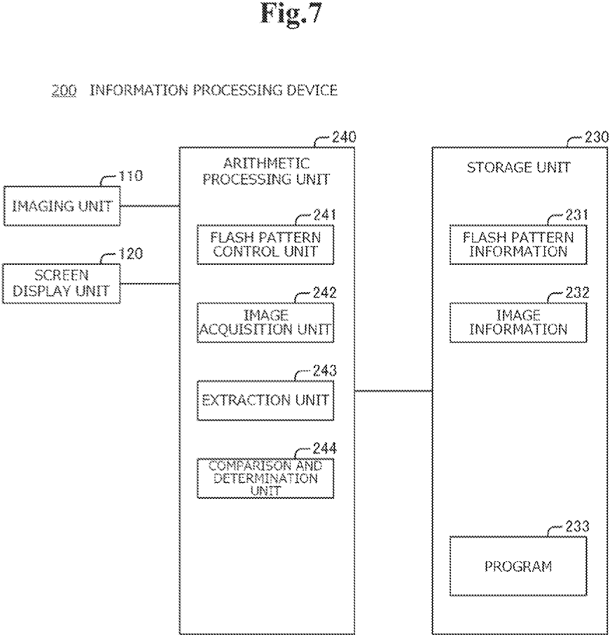

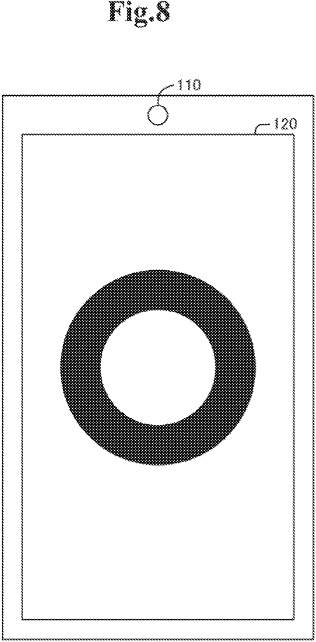

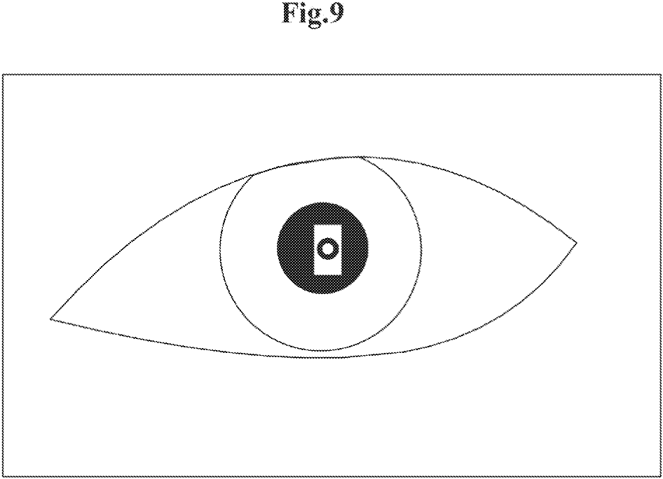

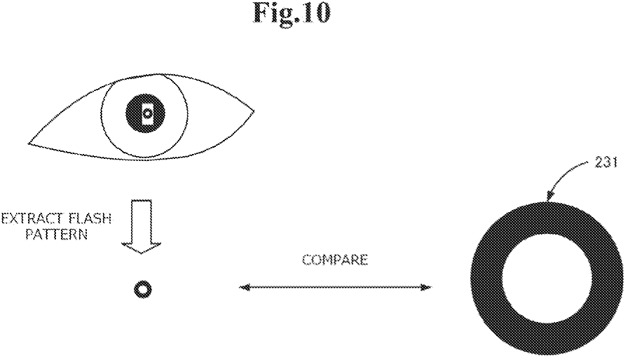

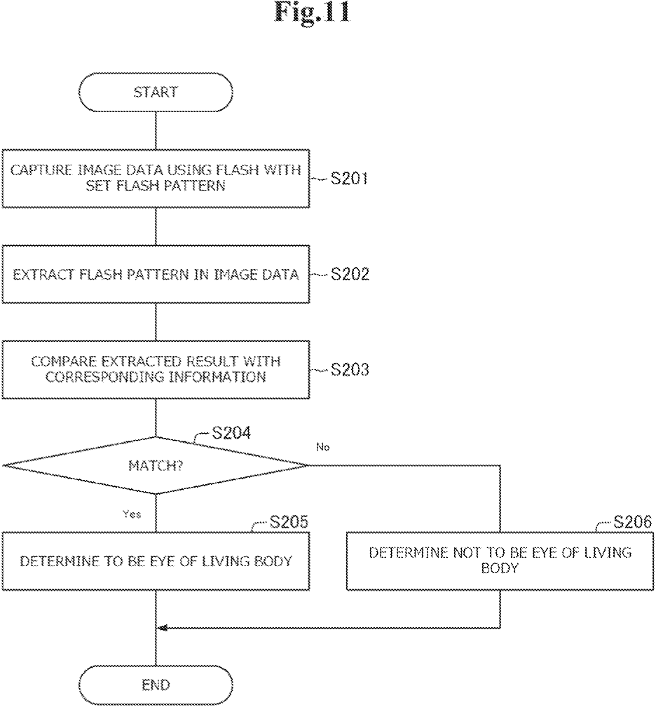

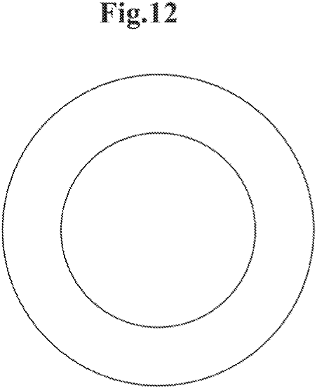

Next, a second exemplary embodiment of the present invention will be described with reference to FIGS. 7 to 12. FIG. 7 is a block diagram illustrating an exemplary configuration of an information processing device 200. FIG. 8 illustrates an example of a flash pattern. FIG. 9 illustrates an example of an eye part in image data. FIG. 10 is an illustration for explaining exemplary processing of an extraction unit 243 and a comparison and determination unit 244. FIG. 11 is a flowchart illustrating an exemplary operation of the information processing device 200. FIG. 12 illustrates another example of a flash pattern.

The second exemplary embodiment of the present invention describes the information processing device 200 that determines whether an imaging object is a living body or one other than a living body such as a photograph, by controlling the shape of a flash. As will be described below, the information processing device 200 controls a flash pattern that is a flash shape control when capturing image data. For example, the information processing device 200 controls a flash pattern by controlling the shape expressed in black color, not in white color, of a flash. Then, the information processing device 200 determines whether or not an imaging object is a living body on the basis of the shape of a flash reflected on image data captured by using a flash whose shape is controlled.

Note that image data acquired by the information processing device 200 can be used for various ways such as an authentication purpose, similar to the case of image data acquired by the information processing device 100.

The information processing device 200 is a device that acquires image data, similar to the case of the information processing device 100. The information processing device 200 of the present embodiment is a mobile terminal such as a smartphone, for example. The information processing device 200 may be a device other than a mobile terminal illustrated as an example.

FIG. 7 illustrates an exemplary configuration of the information processing device 200. Referring to FIG. 7, the information processing device 200 includes, for example, an imaging unit 110, a screen display unit 120, a storage unit 230, and an arithmetic processing unit 240 as constituent elements. The same configurations as those of the information processing device 100 described in the first exemplary embodiment are denoted by the same reference numerals. Hereinafter, characteristic configurations of the present embodiment will be described.

The storage unit 230 is a storage device such as a hard disk or a memory. The storage unit 230 stores therein processing information and a program 233 required for various types of processing executed in the arithmetic processing unit 240. The program 233 is read and executed by the arithmetic processing unit 240 to thereby implement various processing units. The program 233 is read in advance from an external device or a storage medium via the data input/output function of the information processing device 200, and is stored in the storage unit 230. Main information to be stored in the storage unit 230 includes, for example, flash pattern information 231 and image information 232.

The flash pattern information 231 includes information showing a flash pattern. For example, in the flash pattern information 231, information showing a flash pattern and information indicating the time are associated with each other.

Here, information showing a flash pattern will be described in more detail. For example, the first exemplary embodiment has described the case where the screen display unit 120 functions as a flash by displaying a while image in which the luminance value is larger than a predetermined value. In the present embodiment, when the screen display unit 120 functions as a flash, it is possible to show, for example, a black pattern on the screen rather than simply showing white on the entire screen. For example, as illustrated in FIG. 8, when the screen display unit 120 functions as a flash, a black doughnut shape can be shown. As described above, in the present embodiment, when the screen display unit 120 functions as a flash, a flash pattern can be shown. The information showing a flash pattern represents information for specifying a flash pattern that is used when a flash is used, such as a doughnut shape. Note that the flash pattern may be one other than a doughnut shape such as a round shape, a rectangular shape, a triangular shape, or another shape.

The image information 232 includes image data captured by the imaging unit 110 using a flash with a predetermined flash pattern. For example, in the image information 232, image data and information indicating the time that the image data was captured by the imaging unit 110 are associated with each other.

The arithmetic processing unit 240 has a microprocessor such as an MPU and the peripheral circuits thereof, and is configured to read and execute the program 233 from the storage unit 230 to allow the hardware and the program 233 to cooperate with each other to thereby implement the various processing units. Main processing units implemented by the arithmetic processing unit 240 include, for example, a flash pattern control unit 241, an image acquisition unit 242, an extraction unit 243, and a comparison and determination unit 244.

The flash pattern control unit 241 determines a flash pattern when using a flash. In other words, the flash pattern control unit 241 determines the shape that is shown in black on the screen display unit 120 when using a flash, such as a round shape, a rectangular shape, a triangular shape, or a doughnut shape, for example. Then, the flash pattern control unit 241 instructs the screen display unit 120 to use a flash with the determined flash pattern. Further, the flash pattern control unit 241 stores, in the storage unit 230, the determined flash pattern and information indicating the time in association with each other, as flash pattern information 231.

As described above, the flash pattern control unit 241 instructs the screen display unit 120 to make a difference between luminance values in an image to be shown on the screen display unit 120 to thereby realize a flash using the determined flash pattern.

In the present embodiment, the timing of determining and controlling a flash pattern by the flash pattern control unit 241 is not limited particularly. For example, the flash pattern control unit 241 can be configured to determine and control the flash pattern each time image data is acquired by the image acquisition unit 242. alternatively, the flash pattern control unit 241 may be configured to determine and control the flash pattern at predetermined intervals, for example.

Further, in the present embodiment, a method of determining a flash pattern by the flash pattern control unit 241 is not limited particularly. For example, the flash pattern control unit 241 can be configured to determine a flash pattern to be used from among a plurality of predetermined flash patterns. The flash pattern control unit 241 may be configured to generate a flash pattern from time to time, by a well-known algorithm.

Furthermore, the flash pattern control unit 241 may be configured to determine, for example, a flash pattern including an intermediate luminance value, besides a flash pattern expressed in black.

The image acquisition unit 242 instructs the imaging unit 110 to capture image data, and acquires image data from the imaging unit 110. The image acquisition unit 242 also controls a flash using the screen display unit 120. In the present embodiment, it is assumed that a flash is always used when image data is captured for example.

For example, the image acquisition unit 242 instructs the imaging unit 110 to capture image data. The image acquisition unit 242 acquires image data captured in the state of using a flash according to a flash pattern determined by the flash pattern control unit 241. Then, the image acquisition unit 242 stores, in the storage unit 230, the acquired image data in association with the time that the image data was captured by the imaging unit 110, as the image information 232.

The extraction unit 243 extracts a flash pattern from image data. For example, when image data is captured in a state of using a flash using a flash pattern as illustrated in FIG. 8, a flash pattern surrounded by an area corresponding to a flash is reflected on the pupil area as illustrated in FIG. 9. Then, the extraction unit 243 extracts the flash pattern in the image data as illustrated in FIG. 9.

Specifically, for example, the extraction unit 243 extracts a pupil area as similar to the case of the extraction unit 142 described in the first exemplary embodiment. The extraction unit 243 also specifies a flash area corresponding to the flash in the pupil area. Then, the extraction unit 243 extracts a flash pattern in the specified flash area. For example, in the case of FIG. 9, the extraction unit 243 extracts a flash pattern in a doughnut shape that is present in the rectangular flash area as illustrated in FIG. 10.

The comparison and determination unit 244 compares the flash pattern extracted by the extraction unit 243 and the flash pattern information 231. Then, on the basis of a result of comparison, the comparison and determination unit 244 determines whether or not the imaging object is a living body.

For example, the comparison and determination unit 244 acquires, from the extraction unit 243, a flash pattern extracted by the extraction unit 243. The comparison and determination unit 244 also acquires information corresponding to the time that the image data was captured, from the flash pattern information 231. Then, the comparison and determination unit 244 compares the flash pattern acquired from the extraction unit 243 and the corresponding information included in the flash pattern information 231.

As a result of comparison, when the flash pattern acquired from the extraction unit 243 and the flash pattern indicated by the corresponding information included in the flash pattern information 231 match, the comparison and determination unit 244 determines that the imaging object is a living body. On the other hand, when the flash pattern acquired from the extraction unit 243 and the flash pattern indicated by the corresponding information included in the flash pattern information 231 do not match, the comparison and determination unit 244 determines that the imaging object is not a living body.

For example, in the case of FIG. 10, the flash pattern acquired from the extraction unit 243 is in a doughnut shape. The flash pattern indicated by the corresponding information included in the flash pattern information 231 is also in a doughnut shape. Since the flash pattern acquired from the extraction unit 243 and the flash pattern indicated by the corresponding information included in the flash pattern information 231 match, the comparison and determination unit 244 determines that the imaging object is a living body.

The exemplary configuration of the information processing device 200 is as described above. Next, an exemplary operation of the information processing device 200 will be described with reference to FIG. 11.

Referring to FIG. 11, the image acquisition unit 242 of the information processing device 200 acquires image data captured in a state of using a flash according to a flash pattern determined by the flash pattern control unit 241 (step S201).

The extraction unit 243 extracts a flash pattern from the image data (step S202). For example, the extraction unit 243 specifies a flash area corresponding to the flash in the image data, and extracts a flash pattern in the specified flash area.

The comparison and determination unit 244 compares the flash pattern extracted by the extraction unit 243 and the flash pattern indicated by the corresponding information included in the flash pattern information 231 (step S203). When the flash pattern extracted by the extraction unit 243 and the flash pattern indicated by the corresponding information included in the flash pattern information 231 match (step S204, Yes), the comparison and determination unit 244 determines that the imaging object is a living body (step S205). On the other hand, when the flash pattern extracted by the extraction unit 243 and the flash pattern indicated by the corresponding information included in the flash pattern information 231 do not match (step S204, No), the comparison and determination unit 244 determines that the imaging object is not a living body (step S206).

The exemplary operation of the information processing device 200 is as described above.

As described above, the information processing device 200 includes the flash pattern control unit 241 and the comparison and determination unit 244. Further, the information processing device 200 is configured to acquire image data captured in the state of using a flash according to a flash pattern determined by the flash pattern control unit 241. With this configuration, according to a result of comparing the flash pattern extracted by the extraction unit 243 with the flash pattern indicated by the corresponding information included in the flash pattern information 231, the comparison and determination unit 244 can determine whether or not the imaging object is a living body. Thereby, it is possible to easily determine that the object is not a living body when image data such as a photograph is captured and to suppress erroneous authentication with a simple configuration.

Note that the information processing device 200 described in the present embodiment can adopt various modifications as similar to the information processing device 100 described in the first exemplary embodiment. For example, the information processing device 200 may be configured to acquire image data from an external camera or the like having a flash function, or may implement a flash function by that other than the screen display unit 120. Further, the function as the information processing device 200 may be realized by a plurality of information processing devices.

Further, the present embodiment has described the case of controlling a flash pattern by displaying a black pattern on the screen display unit 120 when using a flash. However, a flash pattern may be defined by the shape of a flash itself, for example. For example, as illustrated in FIG. 12, a flash pattern may be defined by using a flash device in a doughnut shape. In the case of using a flash device in a doughnut shape, the comparison and determination unit 244 may be configured to determine whether or not the imaging object is a living body by comparing a predetermined flash pattern corresponding to the shape of the flash device with a flash pattern extracted by the extraction unit 243.

Further, the function as the information processing device 200 described in the present embodiment and the function of the information processing device 100 described in the first exemplary embodiment may be combined to each other. For example, the information processing device may be configured to acquire image data with a flash that is captured with a given flash pattern and image data without a flash. In the case of such a configuration, the information processing device can be configured to determine that the imaging object is a living body when the difference in the ratio of black and white between image data with a flash and image data without a flash is equal to or larger than a comparative threshold and the flash patterns match.

### Third Exemplary Embodiment

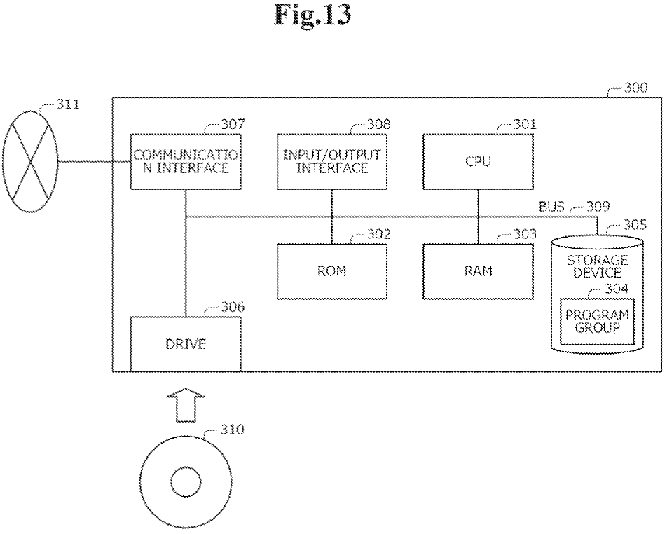

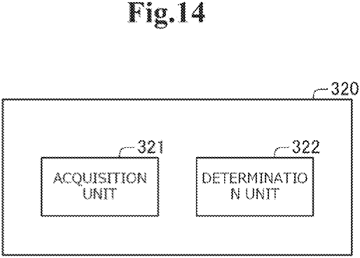

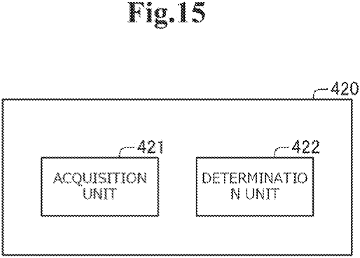

Next, a third exemplary embodiment of the present invention will be described with reference to FIGS. 13 to 15. FIGS. 13 and 14 illustrate exemplary configurations of a determination device 300. FIG. 15 illustrates another exemplary configuration of the determination device 300.

FIG. 13 illustrates an exemplary hardware configuration of the determination device 300. Referring to FIG. 13, the determination device 300 includes a hardware configuration as described below, as an example.
Central Processing Unit (CPU) 301 (arithmetic device)
Read Only Memory (ROM) 302 (storage device)
Random Access Memory (RAM) 303 (storage device)
Program group 304 to be loaded to the RAM 303
Storage device 305 storing therein the program group 304
Drive 306 that performs reading and writing on a storage medium 310 outside the information processing device
Communication interface 307 connecting to a communication network 311 outside the information processing device
Input/output interface 308 for performing input/output of data
Bus 309 connecting the respective constituent elements

Further, the determination device 300 can realize functions as the acquisition unit 321 and the determination unit 322 illustrated in FIG. 14 through acquisition and execution of the program group 304 by the CPU 301. Note that the program group 304 is stored in the storage device 305 or the ROM 302 in advance, and is loaded to the RAM 303 by the CPU 301 as needed. The program group 304 may be provided to the CPU 301 via the communication network 311, or may be stored on a storage medium 310 in advance and read out by the drive 306 and supplied to the CPU 301.

FIG. 13 illustrates an exemplary hardware configuration of the determination device 300. The hardware configuration of the determination device 300 is not limited to that described above. For example, the determination device 300 may be configured of part of the configuration described above, such as without the drive 306.

The acquisition unit 321 acquires image data with a flash captured in a state of using a flash. Further, the acquisition unit 321 acquires image data without a flash captured without using a flash for the same object as that captured in the image data with a flash.

As described above, the acquisition unit 321 acquires image data with a flash and image data without a flash. In the image data with a flash and the image data without a flash, the same imaging object is captured. Acquisition of image data with a flash and acquisition of image data without a flash by the acquisition unit 321 are performed continuously at predetermined intervals, for example.

The determination unit 322 compares the eye area of the imaging object included in the image data with a flash acquired by the acquisition unit 321 with the eye area of the imaging object included in the image data without a flash acquired by the acquisition unit 321. Then, on the basis of a result of comparison, the determination unit 322 determines whether or not the imaging object is a living body.

As described above, the determination device 300 includes the acquisition unit 321 and the determination unit 322. With this configuration, the determination device 300 can determine whether or not the imaging object is a living body on the basis of a comparison result between the image data with a flash and the image data without a flash acquired by the acquisition unit 321. Thereby, it is possible to easily determine that the object is not a living body when image data such as a photograph is captured and to suppress erroneous authentication with a simple configuration.

Note that the determination device 300 described above can be realized by incorporation of a predetermined program in the determination device 300. Specifically, a program that is another aspect of the present invention is a program for implementing, in the determination device 300, the acquisition unit 321 that acquires image data with a flash that is captured in a state of using a flash and image data without a flash that is captured without using a flash for the same imaging object as that captured in the image data with a flash, and the determination unit 322 that determines whether or not the imaging object is a living body by comparing an eye area of the imaging object included in the image data with a flash acquired by the acquisition unit 321 with an eye area of the imaging object included in the image data without a flash acquired by the acquisition unit 321.

Further, a method executed by the determination device 300 is a method of, by the determination device 300, acquiring image data with a flash that is captured in a state of using a flash and image data without a flash that is captured without using a flash for the same imaging object at that captured in the image data with a flash, and determining whether or not the imaging object is a living body by comparing an eye area of the imaging object included in the image data with a flash with an eye area of the imaging object included in the image data without a flash.

Since the invention of a program or a determination method having the above-described configuration also exhibits the same actions and effects as those of the determination device 300, the above-described object of the present invention can be achieved.

Further, the object of the present invention can be realized by a configuration like a determination device 400. Referring to FIG. 15, the determination device 400 includes an acquisition unit 421 and a determination unit 422 for example. Note that the hardware configuration of the determination device 400 may be one similar to the hardware configuration of the determination device 300 described with use of FIG. 13.

The acquisition unit 421 acquires image data captured by using a flash in a state of being controlled such that the shape of the flash becomes a predetermined flash pattern. Then, on the basis of the acquired image data, the determination unit 422 determines whether or not the imaging object is a living body.

As described above, the determination device 400 includes the acquisition unit 421 and the determination unit 422. Even with this configuration, it is possible to easily determine that the object is not a living body when image data such as a photograph is captured and to suppress erroneous authentication with a simple configuration, as similar to the determination device 300. Further, since the invention of a program for realizing the acquisition unit 421 and the determination unit 422 in the determination device 400 or a determination method performed by the determination device 400 also exhibits the same actions and effects as those of the determination device 400, the above-described object of the present invention can be achieved.

### Supplementary Notes

The whole or part of the exemplary embodiments disclosed above can be described as the following supplementary notes. Hereinafter, the outlines of the determination device and the like of the present invention will be described. However, the present invention is not limited to the configurations described below.

### Supplementary Note 1

A determination device comprising:
an acquisition unit that acquires image data with a flash and image data without a flash, the image data with a flash being captured in a state of using a flash, the image data without a flash being image data of an imaging object that is same as an imaging object shown in the image data with a flash and being captured in a state of not using a flash; and
a determination unit that determines whether or not the imaging object is a living body by comparing an eye area of the imaging object included in the image data with a flash with an eye area of the imaging object included in the image data without a flash.

### Supplementary Note 2

The determination device according to supplementary note 1, wherein

the determination unit determines whether or not the imaging object is a living body by comparing a result of binarizing the eye area of the imaging object included in the image data with a flash with a result of binarizing the eye area of the imaging object included in the image data without a flash.

### Supplementary Note 3

The determination device according to supplementary note 1 or 2, wherein

the determination unit determines whether or not the imaging object is a living body on a basis of a difference between a ratio of black and white in an area calculated on a basis of a result of binarizing the eye area of the imaging object included in the image data with a flash and a ratio of black and white in an area calculated on a basis of a result of binarizing the eye area of the imaging object included in the image data without a flash.

### Supplementary Note 4

The determination device according to supplementary note 3, wherein

the determination unit determines that the imaging object is a living body when the difference, between the ratio of black and white in the area calculated on the basis of the result of binarizing the eye area of the imaging object included in the image data with a flash and the ratio of black and white in the the area calculated on the basis of the result of binarizing the eye area of the imaging object included in the image data without a flash, is equal to or larger than a predetermined comparative threshold.

### Supplementary Note 5

The determination device according to supplementary note 3 or 4, wherein

the determination unit determines that the imaging object is not a living body when the difference, between the ratio of black and white in the area calculated on the basis of the result of binarizing the eye area of the imaging object included in the image data with a flash and the ratio of black and white in the area calculated on the basis of the result of binarizing the eye area of the imaging object included in the image data without a flash, is smaller than a predetermined comparative threshold.

### Supplementary Note 6

The determination device according to any of supplementary notes 1 to 5, wherein

the image data with a flash is image data that is captured by using a flash in a state that a shape of the flash becomes a predetermined flash pattern.

### Supplementary Note 7

The determination device according to supplementary note 6, further comprising

an extraction unit that extracts the flash pattern from the image data with a flash, wherein

the determination unit determines whether or not the imaging object is a living body on a basis of the flash pattern extracted by the extraction unit.

### Supplementary Note 8

The determination device according to supplementary note 7, wherein

the determination unit determines that the imaging object is a living body when the flash pattern extracted from the image data with a flash is a predetermined flash pattern.

### Supplementary Note 9

A determination method performed by an information processing device, the method comprising:
acquiring image data with a flash and image data without a flash, the image data with a flash being captured in a state of using a flash, the image data without a flash being image data of an imaging object that is same as an imaging object shown in the image data with a flash and being captured in a state of not using a flash; and
determining whether or not the imaging object is a living body by comparing an eye area of the imaging object included in the acquired image data with a flash with an eye area of the imaging object included in the acquired image data without a flash.

### Supplementary Note 10

A computer-readable storage medium storing thereon a program for causing an information processing device to realize processing to:
acquire image data with a flash and image data without a flash, the image data with a flash being captured in a state of using a flash, the image data without a flash being image data of an imaging object that is same as an imaging object shown in the image data with a flash and being captured in a state of not using a flash; and
determine whether or not the imaging object is a living body by comparing an eye area of the imaging object included in the acquired image data with a flash with an eye area of the imaging object included in the acquired image data without a flash.

It should be noted that the program described in the exemplary embodiments and the supplementary notes may be stored in a storage device or stored on a storage medium readable by a computer. The storage medium is a portable medium such as a flexible disk, an optical disk, a magneto-optical disk, or a semiconductor memory, for example.

While the present invention has been described with reference to the exemplary embodiments described above, the present invention is not limited to the above-described embodiments. The form and details of the present invention can be changed within the scope of the present invention in various manners that can be understood by those skilled in the art.

The present invention is based upon and claims the benefit of priority from Japanese patent application No. 2020-13982, filed on Jan. 30, 2020, the disclosure of which is incorporated herein in its entirety by reference.

### REFERENCE SIGNS LIST

100 information processing device
110 imaging unit
120 screen display unit
130 storage unit
131 flashed image information
132 non-flashed image information
133 program
140 arithmetic processing unit
141 image acquisition unit
142 extraction unit
143 comparison and determination unit
200 information processing device
230 storage unit
231 flash pattern information
232 image information
240 arithmetic processing unit
241 flash pattern control unit
242 image acquisition unit
243 extraction unit
244 comparison and determination unit
300 determination device
301 CPU
302 ROM
303 RAM
304 program group
305 storage device
306 drive
307 communication interface
308 input/output interface
309 bus
310 storage medium
311 communication network
321 acquisition unit
322 determination unit
400 determination device
421 acquisition unit
422 determination unit

What is claimed is:

1. A determination device comprising:
at least one memory configured to store instructions; and
at least one processor configured to execute instructions to:
acquire image data with a flash and image data without a flash, the image data with a flash being captured in a state of using a flash, the image data without a flash being image data of an imaging object that is same as an imaging object shown in the image data with a flash and being captured in a state of not using a flash; and
determine whether or not the imaging object is a living body by comparing an eye area of the imaging object included in the image data with a flash with an eye area of the imaging object included in the image data without a flash.

2. The determination device according to claim 1, wherein the at least one processor is configured to execute the instructions to
determine whether or not the imaging object is a living body by comparing a result of binarizing the eye area of the imaging object included in the image data with a flash with a result of binarizing the eye area of the imaging object included in the image data without a flash.

3. The determination device according to claim 1, wherein the at least one processor is configured to execute the instructions to
determine whether or not the imaging object is a living body on a basis of a difference between a ratio of black and white in an area calculated on a basis of a result of binarizing the eye area of the imaging object included in the image data with a flash and a ratio of black and white in an area calculated on a basis of a result of binarizing the eye area of the imaging object included in the image data without a flash.

4. The determination device according to claim 3, wherein the at least one processor is configured to execute the instructions to
determine that the imaging object is a living body when the difference, between the ratio of black and white in the area calculated on the basis of the result of binarizing the eye area of the imaging object included in the image data with a flash and the ratio of black and white in the the area calculated on the basis of the result of binarizing the eye area of the imaging object included in the image data without a flash, is equal to or larger than a predetermined comparative threshold.

5. The determination device according to claim 3, wherein the at least one processor is configured to execute the instructions to
determine that the imaging object is not a living body when the difference, between the ratio of black and white in the area calculated on the basis of the result of binarizing the eye area of the imaging object included in the image data with a flash and the ratio of black and white in the area calculated on the basis of the result of binarizing the eye area of the imaging object included in the image data without a flash, is smaller than a predetermined comparative threshold.

6. The determination device according to claim 1, wherein
the image data with a flash is image data that is captured by using a flash in a state that a shape of the flash becomes a predetermined flash pattern.

7. The determination device according to claim 6, the at least one processor is configured to execute the instructions to
extract the flash pattern from the image data with a flash, and
determine whether or not the imaging object is a living body on a basis of the flash pattern extracted by the extraction unit.

8. The determination device according to claim 7, wherein the at least one processor is configured to execute the instructions to
determine that the imaging object is a living body when the flash pattern extracted from the image data with a flash is a predetermined flash pattern.

9. A determination method performed by an information processing device, the method comprising:
acquiring image data with a flash and image data without a flash, the image data with a flash being captured in a state of using a flash, the image data without a flash being image data of an imaging object that is same as an imaging object shown in the image data with a flash and being captured in a state of not using a flash; and
determining whether or not the imaging object is a living body by comparing an eye area of the imaging object included in the acquired image data with a flash with an eye area of the imaging object included in the acquired image data without a flash.

10. A non-transitory computer-readable storage medium storing thereon a program comprising instructions for causing an information processing device to realize processing to:
acquire image data with a flash and image data without a flash, the image data with a flash being captured in a state of using a flash, the image data without a flash being image data of an imaging object that is same as an imaging object shown in the image data with a flash and being captured in a state of not using a flash; and
determine whether or not the imaging object is a living body by comparing an eye area of the imaging object included in the acquired image data with a flash with an eye area of the imaging object included in the acquired image data without a flash.
